(19) United States
(12) Patent Application Publication
(10) Pub. No.: US 2011/0295176 A1
(43) Pub. Date: Dec. 1, 2011

(54) VENOUS DEVICE

(76) Inventor: Alfredo R. Zarate, Bethesda, MD (US)

(21) Appl. No.: 12/452,357

(22) PCT Filed: Jun. 25, 2008
(86) PCT No.: PCT/US2008/007866

§ 371 Date: Jun. 14, 2011

Related U.S. Application Data
(60) Provisional application No. 60/947,042, filed on Jun. 29, 2007.

(51) Int. Cl. A61M 1/14; A61M 5/00

(52) U.S. Cl. 604/6.16; 604/246; 604/506

(57) ABSTRACT

A needle can include a fluid directing portion comprising a U-shaped lateral orifice extending between the exterior and interior surfaces and a corresponding U-shaped diverter adjacent to the U-shaped lateral orifice. A diverter can be disposed within a lumen of a needle and transverse to a central axis within the lumen.

### CLAIM OF PRIORITY

This application claims priority to U.S. Provisional Application No. 60/947,042, filed on 29 Jun. 2007, which is hereby incorporated by reference in its entirety.

### TECHNICAL FIELD

The invention relates generally to the field of needles, and has application in the field of dialysis needles.

### BACKGROUND

Hemodialysis with needles requires the use of two needles: one needle called an arterial needle, which suctions blood from the patient (or from the dialysis vascular access) and another needle called a venous needle, which returns blood to the patient. In essence, the process produces blood that has been processed by an artificial kidney. Both needles are inserted into a dialysis vascular access, which can be a surgically modified vein called a fistula or a segment of prosthetic tube (PTFE) inserted between an artery and a vein called a graft.

During the past forty years, since the beginning of two-needle dialysis, many advances in dialysis technology have occurred. Since dialysis with two needles was begun, the dialyzer has increased in efficiency. The volume of blood delivered to the dialyzer to be processed per minute has increased from 200 cc/m to 400-600 cc/m. While this increase in efficiency reduces the amount of time dialysis takes, needle technology has not kept up with the overall level of dialysis technology.

Studies have demonstrated that with conventional or current venous needles, blood exits the needle at high velocity and the mixing of the needle jet with the vein flow causes a high velocity flow and high turbulence. The high velocity of the current venous needle jet and the high turbulence caused by the venous needle jet damages the inside of the vein used for hemodialysis, which has been studied in sheep. In humans, exposure of the vascular access to the high velocity, turbulence and shear stress caused by the venous needle during a dialysis treatment lasting several hours, several times a week, causes new and progressive damage.

The increased blood volume results in an increased flow rate and a proportional increase of the velocity of the blood exiting the venous needle, as well as an increase of the velocity of the flow post-venous needle (needle jet+vein flow) and increased turbulence. The post-venous needle velocity increases proportionally with the increase of the needle flow rate, and the turbulence increases exponentially with the increase of the needle flow rate. In addition, a venous needle jet causes an increase of positive pressure, which facilitates annular recirculation. Using slightly larger needles has decreased the pressure and velocity of the blood jet to a certain extent. However, the use of larger needles can be problematic since larger needles cause greater damage to a patient's skin and blood vessels. Thus, there exists a need for a new venous dialysis needle which will decrease the velocity, turbulence, shear stress and high positive pressure caused by the increased flow of blood.

### SUMMARY

In general, a needle such as a dialysis needle includes a hollow shaft extending from an open proximal end to an open distal end, the shaft having an exterior surface, an interior surface, a lumen, and a central axis, a fluid directing portion comprising a U-shaped lateral orifice extending between the exterior and interior surfaces and a corresponding U-shaped diverter adjacent to the U-shaped lateral orifice. The diverter can be disposed within the lumen and transverse to the central axis. A free end of the diverter can be oriented toward the proximal end of the hollow shaft. An attached end of the diverter can include a slit-shaped opening oriented transverse to the central axis. The needle can include a plurality of fluid directing portions. The plurality of fluid directing portions can include at least two fluid directing portions axially spaced from one another with respect to the central axis. The plurality of fluid directing portions can be evenly spaced around a circumference of said needle. The lateral orifice can have a height-to-width ratio of 1:1-1.25. The diverter can have a height-to-width ratio of 1:1.4-1.8. The distal end of the needle can be blunt and the lateral orifice can be less than 0.7 mm from the open distal end.

The needle can include one row of three fluid directing portions that are spaced about a circumference of the needle by a generally constant angle with respect to the central longitudinal axis. The needle can include a plurality of rows of fluid directing portions axially spaced from one another, the fluid directing portions of each row being spaced about a circumference of the needle by a generally constant angle with respect to the central longitudinal axis. The diverter can have a shape and size substantially the same as the orifice. The diverter can have a shape substantially the same as the orifice. The shaft and diverter can be formed of unitary construction.

The needle can have a distal end that is beveled. The needle can have a lateral orifice spaced at least 0.6 mm from a proximal-most point of the beveled distal end. The needle can have a lateral orifice spaced at least 6 mm from a distal-most point of the beveled distal end. The needle can have a U-shaped lateral orifice that is bevelled.

The needle can include a diverter disposed at an angle less than 40 degrees with respect to the central axis. The needle can include a diverter disposed at an angle less than 38 degrees with respect to the central axis. The diverter can be disposed at an angle less than 36 degrees with respect to the central axis. The diverter can be disposed at an angle less than 32 degrees with respect to the central axis. The diverter can project at least 0.3 mm from the exterior surface toward the central axis. The diverter can project at least 0.5 mm from the exterior surface toward the central axis. The diverter can project at least 0.7 mm from the exterior surface toward the central axis.

A dialysis needle system can include a needle having (1) a hollow shaft having an exterior surface, an interior surface, a lumen, a central axis, an open proximal end, an open distal end, and at least two lateral openings, (2) a U-shaped lateral orifice extending between the exterior and interior surfaces, and (3) a diverter adjacent to the U-shaped lateral orifice and disposed within the lumen, the diverter projecting toward the central axis and having a shape corresponding to the U-shaped lateral orifice, and a trocar sized to be disposed within the hollow shaft.

A fluid delivery system can include an arterial needle, a vascular access, and a venous needle, the venous needle comprising a hollow shaft extending from an open proximal end to an open distal end, the shaft having an exterior surface, an interior surface, a lumen, and a central axis, at least one U-shaped lateral orifice, and a diverter adjacent each U-shaped lateral orifice and disposed within the lumen, the diverter projecting toward the central axis of the hollow shaft and having a shape corresponding to the orifice. The vascular access can be an arteriovenous fistula.

A method of delivering a fluid to a mammal can include removing fluid from the mammal through an arterial needle, passing the fluid through a dialysis vascular access, and returning the fluid to the mammal through a venous needle, the venous needle comprising, a hollow shaft extending from an open proximal end to an open distal end, the shaft having an exterior surface, an interior surface, a lumen, and a central axis, at least one U-shaped lateral orifice, and a diverter adjacent each U-shaped lateral orifice and disposed within the lumen, the diverter projecting toward the central axis of the hollow shaft and having a shape corresponding to the orifice.

A method of delivering a dialyzed fluid to a mammal can include ejecting the dialyzed fluid through an open distal end of a fluid delivery device into a primary flow in a graft, wherein the dialyzed fluid has a velocity of no more than 2.9 meters per second at an average distance of 2 centimeters from the open distal end when measured substantially parallel to a direction of the primary flow in the graft.

### DESCRIPTION OF THE DRAWINGS

FIG. 1 is a partial side view of a needle with a trocar disposed therein, the side view taken parallel to a central longitudinal axis of a needle.

FIG. 2 is a partial side view of another needle and a trocar with alternative distal ends thereof.

FIG. 3 is a partial side view of a distal portion of a needle showing two rows of U-shaped lateral orifices, the side view taken parallel to a central longitudinal axis of a needle.

FIG. 3A is a cross-section of the needle of FIG. 3 taken along a plane 3A-3A perpendicular to a central longitudinal axis of the needle.

FIG. 3B is a cross-section of the needle of FIG. 3 taken along a plane 3B-3B perpendicular to a central longitudinal axis of the needle.

FIG. 4 is a partial side view of a distal portion of a needle showing U-shaped lateral orifices spaced evenly around a circumference of the needle shaft, the side view taken parallel to a central longitudinal axis of a needle.

FIG. 4A is a top perspective of a U-shaped lateral orifice and a corresponding diverter of FIG. 4.

FIG. 5 is a partial side view of a distal portion of a needle showing U-shaped lateral orifices spaced evenly around a circumference of a needle shaft, the side view taken parallel to a central longitudinal axis of a needle.

FIG. 5A is a cross-section of the needle of FIG. 5 taken along a plane 5A-5A perpendicular to a central longitudinal axis of the needle.

FIG. 6 is a partial side view of a distal portion of a needle, the side view taken parallel to a central longitudinal axis of a needle.

FIG. 7 is a partial side view of a distal portion of a needle showing two U-shaped lateral orifices, the side view taken parallel to a central longitudinal axis of a needle.

FIG. 7A is a cross-section of the needle of FIG. 7 taken along a plane 7A-7A perpendicular to a central longitudinal axis of the needle.

FIG. 8 is a partial side view of a distal portion of a needle inserted in a graft.

### DETAILED DESCRIPTION

Prior art needles include needles having a lateral opening disposed on a surface of the needle between proximal and distal ends thereof to improve the flow of fluid out of the needle. However, a lateral opening alone is ineffective because typical fluid dynamics result in fluid flow from the proximal end of the needle to the open distal end without desired flow through the lateral opening.

An improved needle includes a U-shaped lateral orifice extending through an exterior surface and a corresponding diverter. A U-shaped lateral orifice is a type of opening along a length dimension of a needle shaft that is proportional to the size and shape of at least one diverter. A lateral orifice can be a proportionally dimensioned and slightly larger than a diverter. For example, a U-shaped lateral orifice can be a slit that corresponds to the outline of a U-shaped diverter. The U-shaped lateral orifice can be shaped such that a perturbation component of fluid flow is minimized, thereby minimizing turbulent flow. Thus, the U-shaped lateral orifice is dimensioned such that the amount of fluid that escapes through the U-shaped lateral orifice is proportional to the amount of fluid channeled by the diverter. In this manner, flow velocity and pressure is lowered. The U-shaped lateral orifice is also dimensioned and positioned such that the risk of extravasation is minimized and the needle's structural integrity is maintained. In general, a lateral orifice can be positioned 0.3-0.6 mm from an open distal end of a needle, such as a blunt needle or a bevelled needle. Alternatively, a lateral orifice can be positioned less than 3 mm from a distal opening in certain embodiments, such as when the needle is used with a trocar.

A lateral orifice, lateral opening, or diverter can be die-cut or laser cut using standard manufacturing techniques, such as making an indentation in a shaft with a pointed object, by laser cutting, by electrochemical methods, or by abrasive methods, for example. After a needle shaft is formed, a diverter can be formed from the exterior of the shaft, and may be subsequently bent to project into the interior of the needle. Upon bending the diverter into the interior of the needle shaft, the remaining open space can form a lateral orifice. A diverter can be integrally formed with the shaft. Alternatively, a diverter can be added to the shaft and then fused or attached after the shaft is formed.

A lateral orifice is generally oriented such that the U-shape has two sides or legs disposed parallel to the central longitudinal axis of a needle. The legs of the U-shape are joined by an intermediate portion. In certain embodiments, the intermediate portion can be arcuate. In other embodiments, the intermediate portion can be straight.

A needle can have one row of lateral orifices, or two or more rows of lateral orifices. Where a needle has two or more rows of lateral orifices, the lateral orifices can be staggered (for example, a first row can have lateral orifices in 3 and 9 o'clock positions and a second row can have lateral orifices in 12 and 6 o'clock positions) so as not to result on undesired weakening of the needle.

A diverter can be a portion of the shaft that is bent inward, or an additional piece that is attached to the shaft. The diverter can be a non-pivotable diverter positioned adjacent to a lateral orifice and oriented at a fixed angle with respect to a central longitudinal axis disposed in the lumen of a needle. The diverter angle can open away from the distal end of the needle. In other words, the diverter can be disposed to direct fluid out of the lateral orifice, such as by having an orientation transverse to the central longitudinal axis and a free end portion oriented toward a proximal end of the needle. An attached end of the diverter can include a slit-shaped opening oriented transverse to the central axis. This orientation permits blood or other fluid to escape more readily through a lateral orifice while also maintaining the integrity of the diverter angle.

A diverter can be cut from the shaft by any conventional method, and then subsequently trimmed to be slightly smaller than a lateral orifice. A diverter can also be trimmed to be smaller yet proportional to a lateral orifice. Alternatively, a diverter can be previously formed in a desired dimension, and then attached to the interior of a needle shaft. Because a diverter can be cut or formed from a portion of a needle, a diverter may be curved according to an arc of the needle circumference. Alternatively, a diverter may be flat.

Prior art needles are known to have fluid flow problems associated with high pressure jets, and the high velocity of fluid flows result in turbulence and annular recirculation.

Specific openings when combined with specific diverter dimensions and angles positioned along a length dimension of a needle result in decreased velocity, decreased pressure, decreased turbulence, and decreased annular recirculation (and positive pressure), while maintaining the structural integrity of a needle and avoiding extravasation. In one embodiment, a needle can have at least three lateral orifices. In another embodiment, a needle can have a lateral opening of any shape, and at least one U-shaped lateral orifice. In another embodiment, a needle can have two U-shaped lateral orifices, and at least one lateral opening of any shape.

Velocity in centimeters per second (cm/s) is calculated as the flow rate (cm³/s) divided by the area of a graft opening (in cm²). While there is no way to measure an exact value for turbulence directly, both velocity and turbulence can be measured using laser Doppler velocimetry methods. Turbulence is calculated as the root mean square of the fluctuating velocity.

In general, a dialysis system includes an arterial needle that directs fluid away from a subject to a dialyzer, and a venous needle that returns fluid back to the subject. Post-venous needle flow refers to the sum of the needle jet plus the vein or graft flow. Graft flow refers to the typical flow of blood or fluid within a vessel, such as a blood vessel. Generally, velocity and post-venous flow rate depends on the typical flow rate of the graft, the diameter of the graft, the flow rate of the needle, and the diameter of the needle.

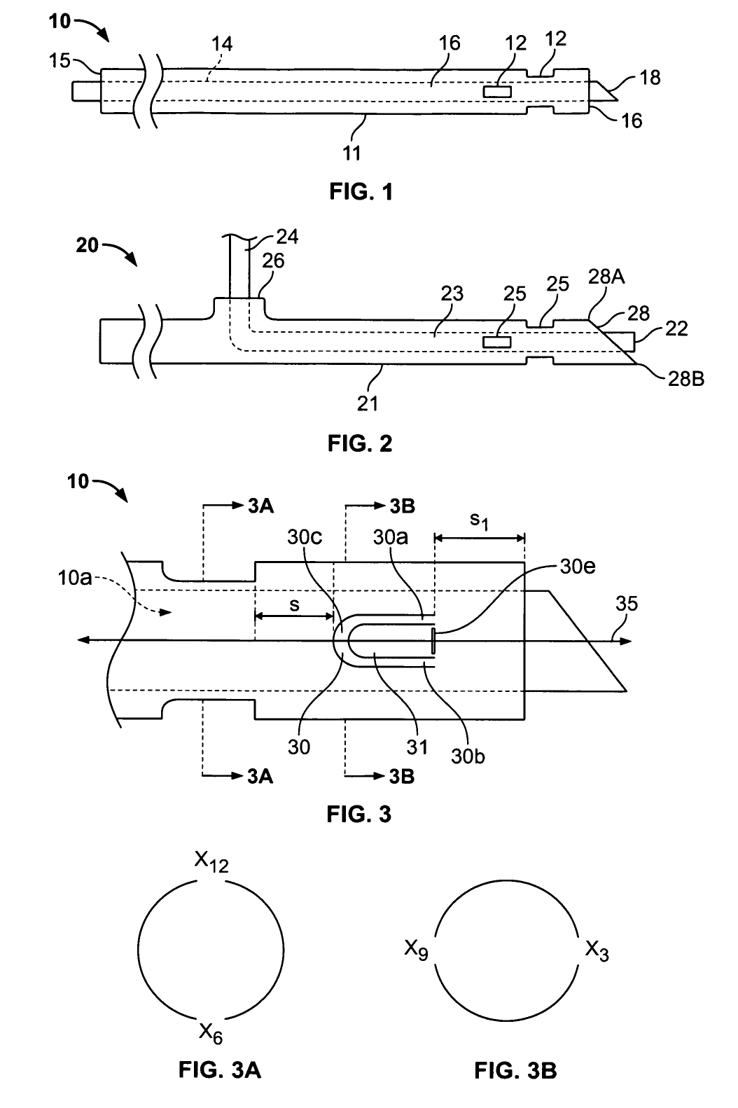

Referring to FIG. 1, a needle 11 can be used with a trocar 14 disposed therein to form a delivery system 10. The needle can have at least one lateral opening 12 through which fluid may be diverted. The needle can have an open proximal end 15 and an open distal end 16. The needle can be used with a trocar 14. The trocar can have a shaft 16 and a distal end 18. The open distal end of the needle can be a blunt end or a bevelled end. An open distal end of a trocar can be a blunt end or a bevelled end.

If it is desired to use a larger number of lateral openings or lateral orifices, it may be advisable to use the needle with a trocar. It can also be advisable to position the lateral openings or lateral orifices in two or more rows, such that the rows are offset, as shown in FIG. 3, so as to not greatly weaken distal end of the needle. The needle can be an arterial needle or a venous needle. The needle can be a 14G, 15G, or 16G needle, for example.

Referring to FIG. 2, a needle 21 can be used with a trocar 24 disposed therein to form a delivery system 20. The distal end 22 of the trocar can be blunt while the distal end of the needle 28 can be bevelled. The trocar may extend through a lateral port 26 in needle 21. The trocar can have a shaft 23 and a distal end 22. The needle can include at least one lateral opening 25 located between 0.8 mm and 2.0 mm from the distal end of the needle shaft. The open distal end 28 of a needle can have a proximal-most point 28A and a distal-most point 28B. The dialysis needle can include a lateral orifice less than 3 mm from the distal end of the needle shaft.

A dialysis needle can have a lateral opening of any shape. A dialysis needle can also have a U-shaped lateral orifice in addition to or in place of a lateral opening.

Referring to FIG. 3, in one embodiment, the needle 10 can have at least one U-shaped lateral orifice 30. The U-shaped lateral orifice can provide communication between an interior 10a of a needle 10 and exterior thereto. The U-shaped lateral orifice 30 can be formed with leg portions 30a, 30b connected by an intermediate portion 30c, which may optionally be arcuate. Leg portions 30a, 30b can be disposed parallel to a central longitudinal axis 39. Optionally, an attached end of the diverter can include a slit-shaped opening 30e oriented transverse to the central axis. A diverter 31 in part can define the U-shaped lateral orifice 30. The diverter 31 can be positioned adjacent to a U-shaped lateral orifice and oriented in a plane transverse to a central longitudinal axis 35. The diverter can be proportional to the size and shape of the U-shaped lateral orifice. For example, diverter can have an arcuate U-shape, and the lateral orifice can have a corresponding arcuate U-shape. Alternatively, a diverter can have a rectangular U-shape, and the lateral orifice can have a corresponding rectangular U-shape. In one embodiment, a U-shaped lateral orifice can be a slit around a diverter. One or more U-shaped lateral orifices can be evenly spaced around the circumference of a needle. As can be seen for example, from FIG. 3, both the orifice 30 and diverter 31 are U-shaped in the sense that the shape defining that boundary of the orifice is a “U” while the shape defining the perimeter of the diverter is also a “U.” In other words, the term “U-shaped” is not limited to defining only the edge of the orifice or the edge of the diverter, but instead is used to describe both.

Referring to FIG. 3A and FIG. 3B, in an exemplary embodiment, a U-shaped lateral orifice can be disposed at position X on the exterior of a needle. For example, if there are two U-shaped lateral orifices, the U-shaped lateral orifices can be disposed at 3 and 9 o'clock positions around the circumference of a needle (designated as X₃ and X₉, respectively). If there are four U-shaped lateral orifices, the U-shaped lateral orifices can be positioned at 12, 3, 6, and 9 o'clock positions (designated as X₁₂, X₆, X₃ and X₉, respectively). If U-shaped lateral orifices are disposed in two or more rows, the U-shaped lateral orifices can be staggered so that the desired needle strength can be maintained. For example, a first row can have two U-shaped lateral orifices at 12 and 6 o'clock positions as indicated in FIG. 3A, and a second row can have two U-shaped lateral orifices at 3 o'clock and 9 o'clock positions as indicated in FIG. 3B. Two or more rows of U-shaped lateral orifices can be positioned such that the rows are separated by a distance s. In an exemplary embodiment, distance s can be 3-6 mm. A U-shaped lateral orifice can be positioned at a distance s₁ from an open distal end of a needle. In an exemplary embodiment distance s₁ can be equal to distance s.

The number of possible fluid jets resulting from flow of fluid from the interior of the needle to the exterior of the needle can vary according to the number of lateral openings or U-shaped lateral orifices on the exterior surface of a needle. Thus, increasing the number of U-shaped lateral orifices can increase the number of jets from a needle. To increase the number of jets, a plurality of lateral openings or U-shaped lateral orifices, or a combination of both can be used. The shape, position, and size of a U-shaped lateral orifice can advantageously divert an amount of fluid, such as blood, for example, at a desired velocity and decrease the risk of recirculation of previously processed blood and the magnitude of turbulence. For example, a U-shaped lateral orifice can divert an amount of fluid at a rate or 0.03-0.06 meters per second.

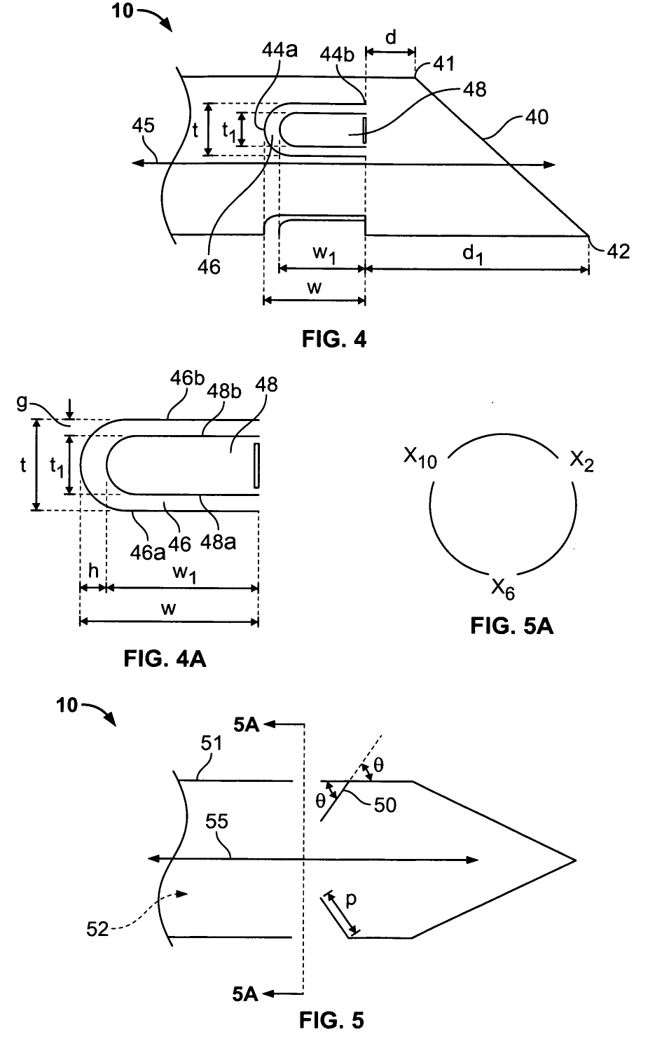

Referring to FIG. 4, in an exemplary embodiment, the needle 10 can include U-shaped lateral orifice 46 disposed at a distance d from an open distal end of a needle shaft. The U-shaped lateral orifice can have a proximal-most point 44a and a distal-most point 44b. The open distal end of a needle shaft can be blunt or bevelled. The distance d can be the distance measured parallel to the central longitudinal axis 45, between a distal-most point 44b of a U-shaped lateral orifice and a proximal-most point 41 of the open distal end 40. The distance d₁ can be the distance measured parallel to the central longitudinal axis 45 from a distal-most point of a U-shaped lateral orifice 44 and a distal-most point of an open distal end 40. If a needle shaft has a blunt end, distance d and distance d₁ can be equal.

With continuing reference to FIG. 4, a U-shaped lateral orifice 46 can have a width dimension w and a height dimension t. A diverter 48 can have a corresponding width dimension w₁ and a corresponding height dimension t₁. In an exemplary embodiment, the U-shaped lateral orifice can be positioned at a distance d of 0.6 mm from the upper border or proximal-most point 41 of the open distal end 40 of a needle. In another exemplary embodiment, the U-shaped lateral orifice can be positioned at a distance d₁ of 6 mm from the lower border or distal-most point 42 of a bevelled opening of a needle. The U-shaped lateral orifice can be positioned less than 6 mm, less than 3 mm, less than 1 mm, less than 0.7 mm, less then 0.4 mm, or less than 0.3 mm from an open distal end 40 of a needle.

Referring to FIG. 4A, diverter 48 can have a maximum width dimension w₁ while lateral orifice 46 can have a maximum width dimension w measured axially with respect to the central longitudinal axis. The diverter can also be disposed adjacent to the U-shaped lateral orifice such that there is a distance h that is the same as or proportional to the distance between the height dimension of the diverter t₁ and the height dimension U-shaped lateral orifice t. The diverter 48 can have generally parallel leg portions 48a, 48b, which in turn are generally parallel to the lateral orifice legs 46a, 46b, with a gap g therebetween.

The U-shaped lateral orifice can have a height-to-width ratio of 1:1, 1:1.25, or 1:1.4. For example, the U-shaped lateral orifice can measure 1.2 mm by 1.2 mm. In another embodiment, the U-shaped lateral orifice can measure 1.2 mm by 1.5 mm. In yet another embodiment, the U-shaped lateral orifice can measure 1.2 mm by 1.7 mm.

The diverter can be dimensioned proportional to the U-shaped lateral orifice. The diverter can also be smaller in size than the U-shaped lateral orifice. The diverter can have a height-to-width ratio of 1:1-1.7, for example. The diverter can measure 0.7 mm by 1.0-1.2 mm.

Referring to FIG. 5, in an exemplary embodiment, a needle 10 can include diverter 50 having an angle θ between an exterior 51 of a needle and a central axis 55 in the lumen interior 52 of a needle. Where there is more than one diverter, the diverters can have the same dimensions, or different dimensions. The diverter can have a length p that projects toward central axis 55 of a needle shaft. The diverter length p can project at least 0.3 mm, at least 0.5 mm, or at least 0.7 mm into the interior of a needle shaft. The diverter can project more than 0.7 mm into the interior of a needle shaft. In an exemplary embodiment, a diverter can be cut from the needle shaft, trimmed to have a U-shape slightly smaller than the U-shaped lateral orifice, and then bent into an interior of the needle shaft, such that the diverter projects toward a central axis in the lumen of the needle shaft. Alternatively, a diverter can be formed and shaped to a desired dimension, subsequently attached to an existing needle shaft, preferably adjacent to a lateral opening therein and then positioned to a desired angle. The diverter can be curved, or alternatively, the diverter can be flat. The curve of a diverter can correspond to an arc of a circumference of the needle shaft.

Referring to FIG. 5A, in an exemplary embodiment, a needle can have three U-shaped lateral orifices and corresponding diverters spaced evenly around the circumference of the needle, for example at 10 o'clock, 2 o'clock and 6 o'clock positions (designated as X₁₀, X₂, and X₆ respectively).

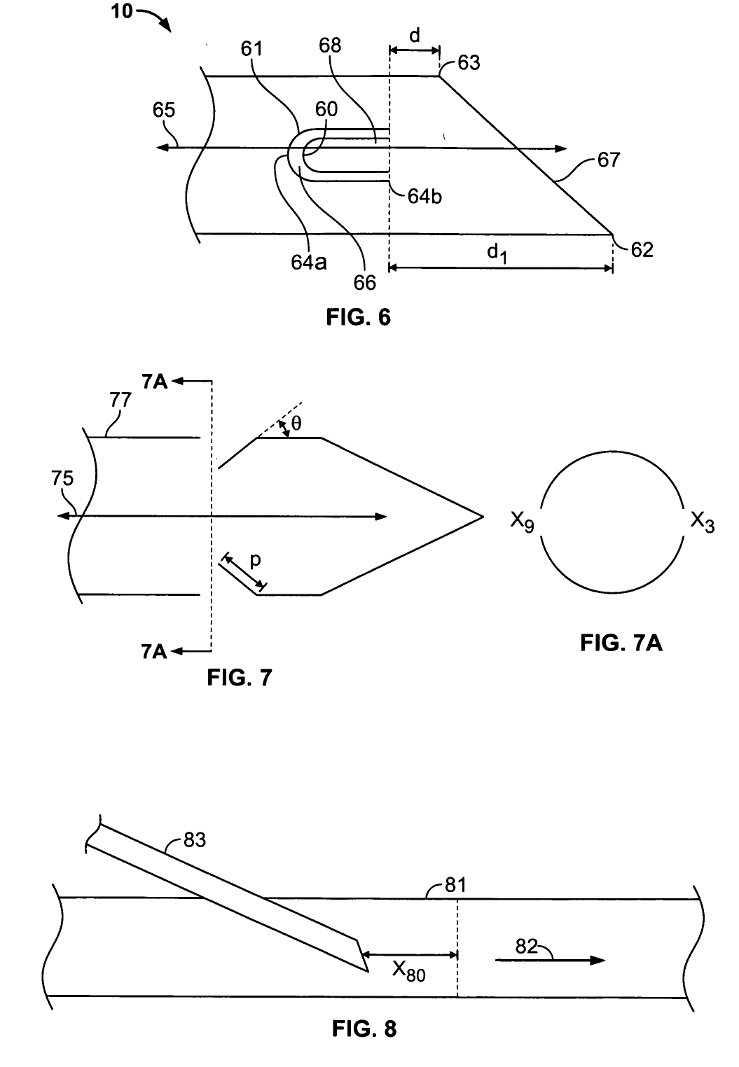

Referring to FIG. 6, in an exemplary embodiment, the diverter can have a U-shape 60 corresponding to a U-shape 61 of a lateral orifice 66. The U-shaped lateral orifice can be positioned adjacent to the diverter 68. The U-shaped lateral orifice can be formed with leg portions connected by intermediate portion 64a, which may optionally be arcuate. The distance d can be the distance, measured parallel to the central longitudinal axis 65, between a distal-most point 64b of a U-shaped lateral orifice and a proximal-most point 63 of the open distal end 67. The distance d₁ can be the distance from a distal-most point 64b of a U-shaped lateral orifice and a distal-most point of an open distal end 65. If a needle shaft is bevelled, distance d₁ will be greater than distance d.

The U-shaped lateral orifice, for example, can be positioned such that distance d is 1 mm measured from the proximal-most point 63 of the distal opening. In another embodiment, the U-shaped lateral orifice can be positioned such that distance d₁ is 6 mm measured from the distal-most point 62 of the distal bevelled opening.

Referring to FIG. 7, in an exemplary embodiment, the diverter can have an angle θ between an exterior plane 77 of a needle and a central axis 75 in the interior of the needle. In certain embodiments, the angle θ of a diverter can be less than 40 degrees, less than 38 degrees, less than 36 degrees, less then 34 degrees, less than 32 degrees, less than 30 degrees, less than 28 degrees, or less than 26 degrees. Also, the diverter can have a lengthp that projects toward a central axis 75 of a needle shaft.

Referring to FIG. 7A, a needle can have two U-shaped lateral orifices and corresponding diverters spaced evenly around the circumference of the needle, for example at 9 o'clock and 3 o'clock positions (designated as X₉ and X₃ respectively).

Referring to FIG. 8, a needle 83 can be inserted in a graft 81. A method of delivering a dialyzed fluid to a mammal can include ejecting the dialyzed fluid through an open distal end of a needle into a primary flow in a graft, wherein the fluid has a velocity measured at an average distance X₈₀ from the open distal end when measured substantially parallel to a direction of the primary flow 82 in the graft. An average distance is the mean distance from the proximal-most point and the distal-most point of an open distal end when measured substantially parallel to a direction of the primary flow in the graft.

When a fluid flow enters through a needle into a graft, such as a blood vessel, the fluid flow produces a jet in the interior of the graft. Jet velocity can result in turbulence. If a jet impacts the wall of a vein, it can cause damage to the tissue, or if too close to the center of the vein, it can result in an area of annular recirculation, which facilitates the recirculation of previously processed blood from the venous needle back through the arterial needle and dialyzer. This is not good for patients since a major purpose of dialysis is to remove impurities from the blood by circulating as much blood as possible through the artificial kidney. If previously processed blood from the venous needle re-enters the arterial needle, it will not allow new “uncleaned blood” to enter the arterial needle. Removing less impurities from the blood, decreases the efficiency of dialysis and less efficient dialysis increases the risk of death.

An exemplary embodiment of an improved needle had a U-shaped lateral orifice and corresponding diverter 0.7 mm in length at 30 degree angle. A conventional needle produces calculated velocities ranging from 2.0 m/s-5.0 m/s. By contrast, this exemplary embodiment of an improved needle produced a calculated velocity of 0.032 m/s.

Flow visualization was carried out to visualize annular recirculation using a fluid dynamic lab with a standard pumping method. Water and glycerine were used to create a mixture having a viscosity was chosen to mimic the viscosity of blood. Indian ink was added to water mixture inside the needle, thereby staining the fluid exiting the needle, and permitting one to visualize post-needle flow. Annular recirculation is visualized according to the intensity of the Indian ink stain. Conventional needles have demonstrated significant turbulence and annular recirculation, By contrast, an exemplary embodiment of an improved needle having three jets demonstrated lower turbulence and no annular recirculation.

Teachings of a general dialysis needle are described in U.S. Pat. No. 5,662,619, which is incorporated by reference herein. A needle as described herein can be advantageously dimensioned to have an angle of greater than 20 degrees and less than 45 degrees, and can have a diverter that is advantageously dimensioned to project more than 0.1 mm into the interior of the shaft. Specifically, a diverter that protrudes 0.35-0.7 mm into the hollow shaft has been shown to divert a significant amount of fluid and to decrease the velocity and turbulence, which can minimize the damage or stress to the graft or blood vessel. Further, a diverter that is less than 40 degrees and greater than 30 degrees has been shown to divert a significant amount of fluid and to decrease the velocity and turbulence which should minimize the damage or stress to the walls of a graft or blood vessel.

Referring to Table 1, a patient's grafts or blood vessels each have a typical velocity of blood flowing through the graft under natural conditions. As indicated, natural blood flow through an average graft (0.6 cm diameter, flow rate of 1,000 mL/min) averages 0.59 m/s (see Table 1, entries 1-4). A conventional dialysis needle, however, exhibits a much higher velocity when it returns blood to a patient (see Table 1, entry 4). As the chart indicates, the needle jet from a conventional needle flows at a velocity up to 3.0-7.0 m/s, which is significantly higher than the typical velocity of fluid or blood in normal vessels. This relatively high velocity causes extremely high turbulence and shear stress that can cause trauma to a surrounding tissue.

TABLE 1

| | |
|---|---|
| | Velocity in meters/ |
| Vessel | second (m/s) |

| | | | |
|---|---|---|---|
| 1 | Saphenous vein | 0.07-0.3 | m/s |
| 2 | Dialysis graft | 0.59 | m/s |
| 3 | Femoral artery | 0.05-0.8 | m/s |
| 4 | Aortic root | 1.5-2.0 | m/s |
| 5 | Vascular access (conventional needle) | 3.0-7.0 | m/s |

As the chart indicates, except in the aorta, velocities of greater than 0.3-0.5 m/s do not occur in normal vessels. Thus, high velocity and turbulence, as is common in prior art needles, can result in damage to vascular access.

A method of delivering a dialyzed fluid to a mammal can include ejecting the dialyzed fluid through an open distal a fluid delivery device into a primary flow in a graft, wherein the fluid has a velocity of no more than 2.9 meters per second at an average distance of 2 centimeters from an open distal end of the needle or fluid delivery device when measured substantially parallel to a direction of the primary flow in the graft. A fluid delivery device can be a venous needle, catheter, or other device for delivering fluid to a mammal. An average distance is the mean distance from the proximal-most point and the distal-most point of the open distal end when measured substantially parallel to a direction of the primary flow in the graft. Fluid can be delivered for 2-7 hours. For example, the fluid can be ejected from a fluid delivery device for at least 2 hours, at least 3 hours, at least 4 hours, at least 5 hours, or at least 6 hours.

A number of embodiments have been described. Nevertheless, it will be understood that various modifications may be made without departing from the spirit and scope of the invention. Accordingly, other embodiments are within the scope of the following claims.

1. A dialysis needle comprising:
a hollow shaft extending from an open proximal end to an open distal end, the shaft having an exterior surface, an interior surface, a lumen, and a central axis;
a fluid directing portion comprising a U-shaped lateral orifice extending between the exterior and interior surfaces and a corresponding U-shaped diverter adjacent to the U-shaped lateral orifice;
wherein the diverter is disposed within the lumen and transverse to the central axis.

2. The dialysis needle of claim 1, wherein a free end of the diverter is oriented toward the proximal end of the hollow shaft.

3. The dialysis needle of claim 1, wherein an attached end of the diverter includes a slit-shaped opening oriented transverse to the central axis.

4. The dialysis needle of claim 1, wherein the needle comprises a plurality of fluid directing portions.

5. The dialysis needle of claim 4, wherein the plurality of fluid directing portions comprises at least two fluid directing portions axially spaced from one another with respect to the central axis.

6. The dialysis needle of claim 4, wherein the plurality of fluid directing portions are evenly spaced around a circumference of said needle.

7. The dialysis needle of claim 1, wherein the lateral orifice has a height-to-width ratio of 1:1-1.25.

8. The dialysis needle of claim 1, wherein the diverter has a height-to-width ratio of 1:1.4-1.8.

9. The dialysis needle of claim 1, wherein the distal end of the needle is blunt and the lateral orifice is less than 0.7 mm from the open distal end.

10. The dialysis needle of claim 1, wherein the needle comprises one row of three fluid directing portions that are spaced about a circumference of the needle by a generally constant angle with respect to the central longitudinal axis.

11. The dialysis needle of claim 1, wherein the needle comprises a plurality of rows of fluid directing portions axially spaced from one another, the fluid directing portions of each row being spaced about a circumference of the needle by a generally constant angle with respect to the central longitudinal axis.

12. The dialysis needle of claim 1, wherein the diverter has a shape and size substantially the same as the orifice.

13. The dialysis needle of claim 1, wherein the diverter has a shape substantially the same as the orifice.

14. The dialysis needle of claim 1, wherein the shaft and diverter are formed of unitary construction.

15. The dialysis needle of claim 1, wherein said distal end is beveled.

16. The dialysis needle of claim 15, wherein the lateral orifice is spaced at least 0.6 mm from a proximal-most point of the beveled distal end.

17. The dialysis needle of claim 15, wherein the lateral orifice is spaced at least 6 mm from a distal-most point of the beveled distal end.

18. The dialysis needle of claim 1, wherein the U-shaped lateral orifice is bevelled.

19. The dialysis needle of claim 1, wherein the diverter is disposed at an angle less than 40 degrees with respect to the central axis.

20. The dialysis needle of claim 1, wherein the diverter is disposed at an angle less than 38 degrees with respect to the central axis.

21. The dialysis needle of claim 1, wherein the diverter is disposed at an angle less than 36 degrees with respect to the central axis.

22. The dialysis needle of claim 1, wherein the diverter is disposed at an angle less than 32 degrees with respect to the central axis.

23. The dialysis needle of claim 1, wherein the diverter projects at least 0.3 mm from the exterior surface toward the central axis.

24. The dialysis needle of claim 1, wherein the diverter projects at least 0.5 mm from the exterior surface toward the central axis.

25. The dialysis needle of claim 1, wherein the diverter projects at least 0.7 mm from the exterior surface toward the central axis.

26. A dialysis needle system comprising:
a needle having (1) a hollow shaft having an exterior surface, an interior surface, a lumen, a central axis, an open proximal end, an open distal end, and at least two lateral openings, (2) a U-shaped lateral orifice extending between the exterior and interior surfaces, and (3) a diverter adjacent to the U-shaped lateral orifice and disposed within the lumen, the diverter projecting toward the central axis and having a shape corresponding to the U-shaped lateral orifice; and
a trocar sized to be disposed within the hollow shaft.

27. A fluid delivery system comprising:
an arterial needle;
a vascular access;
a venous needle, the venous needle comprising:
a hollow shaft extending from an open proximal end to an open distal end, the shaft having an exterior surface, an interior surface, a lumen, and a central axis;
at least one U-shaped lateral orifice; and
a diverter adjacent each U-shaped lateral orifice and disposed within the lumen, the diverter projecting toward the central axis of the hollow shaft and having a shape corresponding to the orifice.

28. The system of claim 27, wherein the vascular access is an arteriovenous fistula.

29. A method of delivering a fluid to a mammal comprising:
removing fluid from the mammal through an arterial needle;
passing the fluid through a dialysis vascular access; and
returning the fluid to the mammal through a venous needle, the venous needle comprising:
a hollow shaft extending from an open proximal end to an open distal end, the shaft having an exterior surface, an interior surface, a lumen, and a central axis;
at least one U-shaped lateral orifice; and
a diverter adjacent each U-shaped lateral orifice and disposed within the lumen, the diverter projecting toward the central axis of the hollow shaft and having a shape corresponding to the orifice.

30. A method of delivering a dialyzed fluid to a mammal comprising:
ejecting the dialyzed fluid through an open distal end of a fluid delivery device into a primary flow in a graft, wherein the dialyzed fluid has a velocity of no more than 2.9 meters per second at an average distance of 2 centimeters from the open distal end when measured substantially parallel to a direction of the primary flow in the graft.

31. The method of claim 30, wherein the dialyzed fluid is ejected for at least 2 hours.

32. The method of claim 30, wherein the dialyzed fluid is ejected for at least 3 hours.

33. The method of claim 30, wherein the dialyzed fluid is ejected for at least 4 hours.

34. The method of claim 30, wherein the dialyzed fluid is ejected for at least 5 hours.

35. The method of claim 30, wherein the dialyzed fluid is ejected for at least 6 hours.

36. The method of claim 30, wherein the fluid delivery device comprises:
a hollow shaft extending from an open proximal end to an open distal end, the shaft having an exterior surface, an interior surface, a lumen, and a central axis;
at least one U-shaped lateral orifice; and
a diverter adjacent each U-shaped lateral orifice and disposed within the lumen, the diverter projecting toward the central axis of the hollow shaft and having a shape corresponding to the orifice.
